(19) United States
(12) Patent Application Publication
(10) Pub. No.: US 2004/0009151 A1
(43) Pub. Date: Jan. 15, 2004

(54) Methods for delivering recombinant adeno-associated virus virions to the liver of a mammal

(76) Inventors: Mark A. Kay, Los Altos, CA (US); Katherine A. High, Merion Station, PA (US); Linda B. Couto, Pleasanton, CA (US)

Correspondence Address: STOEL RIVES LLP, 201 SOUTH MAIN STREET, SUITE 1100, SALT LAKE CITY, UT, 84111, US

(21) Appl. No.: 10/405,047

(22) Filed: Apr. 1, 2003

Related U.S. Application Data
(60) Provisional application No. 60/370,061, filed on Apr. 4, 2002.

(51) Int. Cl. A61K 48/00; C12N 15/861

(52) U.S. Cl. 424/93.2; 435/456

(57) ABSTRACT

Methods for introducing recombinant adeno-associated virus (rAAV) virions into the liver of a mammal are provided. In these methods, the liver is partially or completely isolated from its blood supply, a catheter is introduced into the liver via a peripheral blood vessel, and rAAV virions are then infused through the catheter to the liver. The methods described herein may be used, for example, to deliver heterologous genes encoding therapeutic proteins to the hepatocytes of humans. This can be accomplished, for example, by introducing the catheter into a femoral artery, threading the catheter into the hepatic artery, and infusing rAAV virions through the catheter and into the liver. Exemplary examples of heterologous genes include those coding for blood coagulation factors.

### CROSS REFERENCE TO RELATED APPLICATIONS

This application claims priority under 37 U.S.C. § 119(e) to Provisional Application Ser. No. 60/370,061 filed on Apr. 4, 2002.

### GOVERNMENT SUPPORT

This invention was supported in part by grants from the U.S. Government (NIH Grant Nos. R01 HL53682, R01 HL53688, R01 HL61921, and P50 HL54500) and the U.S. Government may therefore have certain rights in the invention.

### FIELD OF THE INVENTION

The present invention relates to methods of delivering rAAV virions to a mammal. More specifically, the present invention relates to methods of delivering recombinant adeno-associated virus (rAAV) virions to a target organ of a mammal by use of a catheter.

### BACKGROUND

Non-Systemic Delivery of Therapeutic Substances Therapeutic agents are frequently administered via oral and intravenous methods. However, because these delivery routes distribute drugs throughout the body, they are not ideal in situations where the drug is needed only at a particular target site. In such cases, it is often necessary to increase drug dosage in order to achieve therapeutic efficacy. Increasing dosage, however, increases the likelihood of eliciting unwanted effects, toxic and otherwise. Therefore, in many cases it is desirable to identify and employ a method of delivery that reduces or even eliminates systemic drug distribution.

Improved drug delivery techniques have been developed in response to the limitations discussed above, with significant research and development efforts aimed at delivering chemotherapeutic drugs directly to cancerous tissue, thus reducing or eliminating exposure to healthy tissue. One such procedure, isolated liver perfusion, was developed to more effectively treat liver cancer patients, especially those patients with unresectable tumors. Early isolated liver perfusion efforts focused on partially isolating the liver from the systemic circulation, principally by occluding venous flow from the liver; arterial blood flow, however, was not isolated. In this procedure, the blood vessels of the liver are accessed via an abdominal incision, and blood outflow is controlled by ligation of the hepatic veins. A catheter is used to deliver a chemotherapeutic agent to the liver by threading it into an artery supplying the liver (such as the hepatic artery). Outflow is then rerouted to a filtration system that effectively removes excess drug from the bloodstream. This method facilitates an increase in drug concentration at the target site while decreasing circulating drug concentration, thereby increasing therapeutic efficacy and decreasing toxicity.

While such methods permit organ isolation, they are highly invasive and may not be warranted for applications that treat non-life-threatening conditions—especially diseases where other treatment options are available. As a result, less invasive surgical procedures for delivering therapeutic agents to organs and tissues have also been designed. Such methods involve infusion by means of a catheter inserted into a peripheral blood vessel, which is then threaded to the primary vessels entering the tissue or organ of interest.

Gene Therapy

Scientists are continually discovering genes that are associated with human diseases such as diabetes, hemophilia, and cancer. Research efforts have also uncovered genes, such as erythropoietin (which increases red blood cell production), that are not associated with genetic disorders but instead code for proteins that can be used to treat numerous diseases. Despite significant progress in the effort to identify and isolate genes, however, a major obstacle facing the biopharmaceutical industry is how to safely and persistently deliver therapeutically effective quantities of gene products to target sites, a situation analogous to the problem of chemotherapeutic drug delivery described above.

Generally, the protein products of these genes are synthesized in cultured bacterial, yeast, insect, mammalian, or other cells and delivered to patients by direct injection. Injection of recombinant proteins has been successful but suffers from several drawbacks. For example, patients often require weekly, and sometimes daily, injections in order to maintain the necessary levels of the protein in the bloodstream. Even then, the concentration of protein is not maintained at physiological levels—the level of the protein is usually abnormally high immediately following the injection, and far below optimal levels prior to the injection. Additionally, injecting recombinant protein is often not successful in delivering the protein to the target cells, tissues, or organs of the body. And, if the protein reaches its target, it is often diluted to non-therapeutic levels, which may require increasing the dose of the protein in order to achieve a therapeutic effect; however, like the situation with chemotherapy, this approach has its shortcomings, as increasing the dose of recombinant protein can lead to toxicity. Furthermore, the method is inconvenient and severely restricts the patient's lifestyle.

These shortcomings have led to the development of gene therapy methods for delivering sustained levels of specific proteins into patients. These methods are designed to allow clinicians to introduce deoxyribonucleic acid (DNA) coding for a heterologous nucleic acid molecule (HNA) directly into a patient (in vivo gene therapy) or into cells isolated from a patient or a donor (ex vivo gene therapy). The introduced DNA then directs the patient's own cells or grafted cells to produce the desired protein product. Gene delivery, therefore, obviates the need for frequent injections. Gene therapy may also allow clinicians to select specific organs or cellular targets (e.g., muscle, blood cells, brain cells, liver, etc.) for therapy.

DNA may be introduced into a patient's cells in several ways. There are transfection methods, including chemical methods such as calcium phosphate precipitation and liposome-mediated transfection, and physical methods such as electroporation. There are also methods that use recombinant viruses. Current viral-mediated gene delivery vectors include those based on retrovirus, adenovirus, herpes virus, pox virus, and adeno-associated virus (AAV). Like the retroviruses, and unlike adenovirus, AAV has the ability to integrate its genome into a host cell chromosome.

Adeno-Associated Virus

AAV, a parvovirus belonging to the genus Dependovirus with eight known serotypes (designated AAV-1 through AAV-8), has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing an HNA, a suitable host cell line is transfected with an AAV vector containing the HNA, but lacking rep and cap. The host cell is then infected with wild-type (wt) AAV and a suitable helper virus to form rAAV virions. Alternatively, wt AAV genes (also known as AAV helper function genes, comprising rep and cap) and helper virus function genes (also known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the therapeutic gene. The gene of interest is then replicated and packaged as though it were a wt AAV genome, forming a recombinant AAV virion. When a patient's cells are transduced with the resulting rAAV virion, the gene enters and is expressed in the patient's cells. Because the patient's cells lack the rep and cap genes, as well as the accessory function genes, the rAAV virion cannot further replicate and package its genomes. Moroever, without a source of rep and cap genes, wt AAV virions cannot be formed in the patient's cells.

As a gene delivery vector, AAV has many desirable qualities, including minimum pathogenicity (or non-pathogenicity), the ability to transduce non-dividing cells, the ability to integrate into a host cell chromosome, prolonged heterologous gene expression, etc. If AAV is administered systemically, however, some of these desirable qualities may, in certain circumstances, be disadvantageous. For instance, systemic administration (e.g., intravenous administration) may result in unwanted transduction, which can potentially lead to adverse effects such as increased transduction of antigen presenting cells and the development of an immune response to the therapeutic protein expressed from the heterologous gene, or it may require the administration of higher viral doses to overcome a potential dilution effect and achieve sufficient transduction efficiency (and hence, therapeutic levels of protein expression). Furthermore, because of the potential for the induction of an immune response to AAV administered systemically, transduction efficiency is greatly reduced upon subsequent readministration or in individuals previously exposed to AAV.

Systemic administration leading to widespread tissue transduction could complicate the ability to treat the patient should unwanted toxicity occur. If several tissues and/or organs are transduced and excision of transduced tissues/organs becomes necessary to treat recombinant AAV-associated toxicity, then having multiple sites of transduction may make it impossible to treat any toxic effects by removal of transduced tissue.

Thus, there remains an unmet medical need to provide methods that safely and reliably deliver therapeutic levels of genes and/or gene products to specific target organs and tissues in the patient. It would be an advancement in the art to provide methods to deliver genes and/or gene products to a target organ or a portion of the target organ (e.g., glucocerebrosidase to macrophages residing in the sinusoids of the liver) to facilitate site-specific gene expression. Furthermore, it would be a significant advancement in the art if methods were available to isolate the target organ or portions of the target organ from systemic blood circulation, wherein the recombinant viral virion carrying the heterologous gene is not exposed to circulating antibodies, wherein the gene and/or gene products can be concentrated at the site of action, and wherein the recombinant viral virion is localized to a certain organ, or to a specific component of the organ, so that the ability to treat any adverse side effects is facilitated.

Such methods are disclosed herein.

### SUMMARY OF THE INVENTION

The present invention relates to methods of delivering rAAV virions to a target organ of a mammal. In one embodiment, the method includes the steps of (1) introducing a catheter into the liver, and (2) infusing the rAAV virions through the catheter into the liver. In certain embodiments, the catheter is introduced via a blood vessel. In other embodiments, the catheter is introduced via an artery, e.g., , the rAAV virions may be introduced into the liver by catheter via the hepatic artery. Introduction to the liver can be accomplished while the liver is partially or completely isolated from the systemic circulation.

The rAAV virions may comprise a heterologous nucleic acid molecule that codes for a protein to be expressed in the target organ. Such rAAV virions may be used to introduce genetic material into mammals, including humans, for a variety of research and therapeutic uses. For example, rAAV virions of the present invention may be used to express DNA encoding a protein, anti-sense RNA, or a ribozyme in animals to gather preclinical data or to screen for potential drug candidates. Alternatively, the rAAV virions may be used to transfer genetic material into the liver of a human to treat or cure a genetic defect or to effect a desired treatment.

In another embodiment, the present invention provides methods of introducing rAAV virions into the liver of a mammal comprising the steps of (1) creating an access site in the femoral artery of the mammal, (2) introducing a guidewire into the femoral artery, (3) introducing a sheath over the guidewire into the femoral artery, (4) advancing a catheter through the sheath into the hepatic artery, (5) infusing the rAAV virions through the catheter into the hepatic artery, (6) removing the catheter and the sheath from the femoral artery, and (7) repairing the access site. In a preferred embodiment, the mammal is a human. The rAAV virions may contain a gene that codes for a therapeutic protein, e.g., a coagulation factor, such as Factor VIII or Factor IX, or a gene that codes for an enzyme involved in metabolism such as glucocerebrosidase. Alternatively, the rAAV virions may contain a gene encoding a protein having an anti-cancer therapeutic effect, e.g., a cytokine or a tumor antigen.

The present invention also provides methods of treating a disease in a human, comprising delivering rAAV virions into the liver of the human by injection into the hepatic artery, wherein the rAAV virions contain a heterologous gene coding for a protein, such that cells within the liver express the heterologous gene at a level providing a therapeutically effective concentration of the protein in the human. In certain embodiments, the heterologous gene codes for the light and heavy chains of Factor VIII. In certain other embodiments, the heterologous gene codes for a Factor VIII protein lacking a portion of the B-domain region. In certain other embodiments, the heterologous gene codes for Factor IX. In certain other embodiments, the heterologous gene codes for glucocerebrosidase.

These and other advantages of the present invention will become apparent upon reading the following detailed description and appended claims.

### BRIEF DESCRIPTION OF THE DRAWINGS

FIG. 1 is a photograph of the hepatic artery catheter after it was inserted into the femoral artery, advanced through the aorta, and then directed into the hepatic artery. The arrow denotes the tip of the hepatic artery catheter after it has been advanced into the hepatic artery. Contrast dye was infused to visualize the arterial vascular bed.

FIG. 2 is a picture of a 0.8% agarose gel showing restriction digests of DNA extracted from two different liver lobes of dogs three to four months after administration of 5×10¹² vector genomes/kg body weight of rAAV-null (non-functional expression cassette). For details of methods, see Example 1, infra.

FIG. 3 is a schematic showing portal vein and hepatic artery infusion into selected lobes of the rat liver. The top schematic shows hepatic artery infusion alone. The bottom left schematic shows selective hepatic artery infusion with clamped blood vessels shown with rectangles. By this method selective perfusion of the caudate and left liver lobes is achieved. The bottom right schematic shows portal vein infusion with clamping of specific arterial branches allowing for selective perfusion of the caudate lobes. Abbreviations: PHA—proper hepatic artery; CHA—common hepatic artery; GDA—gastroduodenal artery; MPV—main portal vein.

FIG. 4 is a photograph depicting infusion of selected hepatic lobes by hepatic artery (left) or portal vein (right). The dark coloration of the liver lobes results from the infusion of the dye. Non-stained liver lobes remain light in color.

FIG. 5 is a schematic of the asanguineuos hepatic perfusion (AHP) method. The figure on the left depicts a catheter for liver bypass shunting. The figure on the right depicts the circuit for AHP. See text for details of AHP methodology.

### DETAILED DESCRIPTION OF THE INVENTION

The present invention is directed to novel methods for introducing rAAV virions comprising a heterologous nucleic acid molecule of interest into a target organ of a mammal. By “heterologous” is meant a nucleic acid molecule flanked by nucleotide sequences not found in association with the nucleic acid molecule in nature. Alternatively, “heterologous” embraces the concept of a nucleic acid molecule that itself is not found in nature (e.g., synthetic sequences having codons different from a native gene). Allelic variation or naturally occurring mutational events do not give rise to heterologous nucleic acid molecules, as used herein. Nucleic acid molecules can be in the form of genes, promoters, enhancers, or any other nucleic acid-containing molecule so long as they adhere to the definition of “heterologous,” as used herein.

By “AAV virion” is meant a complete virus particle, such as a wtAAV particle (comprising a linear, single-stranded AAV nucleic acid genome associated with an AAV capsid protein coat). In this regard, single-stranded AAV nucleic acid molecules of either complementary sense, i.e., “sense” or “antisense” strands, can be packaged into any one AAV virion and both strands are equally infectious. A “recombinant AAV virion” or “rAAV virion” is defined herein as an infectious virus composed of an AAV protein shell (i.e., capsid) encapsulating a heterologous nucleic acid molecule that is flanked on both sides by AAV ITRs. The nucleotide sequences of AAV ITR regions are known. See, e.g., Kotin, (1994) Human Gene Therapy 5:793-801; Berns, K. I. “Parvoviridae and their Replication” in Fundamental Virology, 2d ed., (B. N. Fields and D. M. Knipe, eds.) for the AAV-2 sequence. As used herein, an “AAV ITR” need not have the wild-type nucleotide sequence depicted in these references, but may be altered, e.g., by the insertion, deletion, or substitution of nucleotides. Additionally, the AAV ITR may be derived from any of several AAV serotypes, including without limitation, AAV-1, AAV-2, AAV-3A, AAV-3B, AAV-4, AAV-5, AAV-6, AAV-7, AAV-8, etc. Furthermore, 5′ and 3′ ITRs, which flank a selected nucleotide sequence in an AAV vector, need not necessarily be identical or derived from the same AAV serotype or isolate, so long as they function as intended, i.e., to allow for excision and rescue of the sequence of interest from a host cell genome when AAV Rep gene products are present in the cell.

Recombinant AAV virions may be produced using a variety of art-recognized techniques. For example, the skilled artisan can use wt AAV and helper viruses to provide the necessary replicative functions for producing rAAV virions (see, e.g., U.S. Pat. No. 5,139,941, herein incorporated by reference). Alternatively, a plasmid, containing helper function genes, in combination with infection by one of the well-known helper viruses can be used as the source of replicative functions (see e.g., U.S. Pat. No. 5,622,856, herein incorporated by reference; U.S. Pat. No. 5,139,941, supra). Similarly, the skilled artisan can make use of a plasmid, containing accessory function genes, in combination with infection by wt AAV, to provide the necessary replicative functions. As is familiar to one of skill in the art, these three approaches, when used in combination with a rAAV vector, are each sufficient to produce rAAV virions. Other approaches, well known in the art, can also be employed by the skilled artisan to produce rAAV virions.

In a preferred embodiment of the present invention, the triple transfection method (described in detail in U.S. Pat. No. 6,001,650, the entirety of which is incorporated by reference) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV vector. One of skill in the art will appreciate, however, that the nucleic acid sequences encoded by these vectors can be provided on two or more vectors in various combinations. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The AAV helper function vector encodes the “AAV helper function” sequences (i.e., rep and cap), which function in trans for productive AAV replication and encapsidation. Preferably, the AAV helper function vector supports efficient AAV vector production without generating any detectable wt AAV virions (i.e., AAV virions containing functional rep and cap genes). An example of such a vector, pHLP19 is described in U.S. Pat. No. 6,001,650, supra, and in Example 1, infra. The rep and cap genes of the AAV helper function vector can be derived from any of the known AAV serotypes. For example, the AAV helper function vector may have a rep gene derived from AAV-2 and a cap gene derived from AAV-6; one of skill in the art will recognize that other rep and cap gene combinations are possible, the defining feature being the ability to support rAAV virion production.

The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno5 is used (details regarding pLadeno5 are described in U.S. Pat. No. 6,004,797, the entirety of which is hereby incorporated by reference). This plasmid provides a complete set of adenovirus accessory functions for AAV vector production, but lacks the components necessary to form replication-competent adenovirus.

The rAAV vector can be a vector derived from any AAV serotype, including without limitation, AAV-1, AAV-2, AAV-3A, AAV-3B, AAV-4, AAV-5, AAV-6, AAV-7, AAV-8, etc. AAV vectors can have one or more of the wt AAV genes deleted in whole or in part, i.e., the rep and/or cap genes, but retain at least one functional flanking ITR sequence, as necessary for the rescue, replication, and packaging of the AAV virion. Thus, an AAV vector is defined herein to include at least those sequences required in cis for viral replication and packaging (e.g., functional ITRs). The ITRs need not be the wild-type nucleotide sequences, and may be altered, e.g., by the insertion, deletion, or substitution of nucleotides, so long as the sequences provide for functional rescue, replication, and packaging. AAV vectors can be constructed using recombinant techniques that are known in the art to include one or more heterologous nucleic acid molecules flanked with functional AAV ITRs.

The heterologous nucleic acid molecule is operably linked to a heterologous promoter (constitutive, cell-specific, or inducible) such that the gene is capable of being expressed in the patient's target cells under appropriate or desirable conditions. By “operably linked” is meant an arrangement of elements wherein the components so described are configured so as to perform their usual function. Thus, control sequences operably linked to a coding sequence are capable of effecting the transcription of the coding sequence. The control sequences need not be contiguous with the coding sequence, so long as they function to direct the transcription thereof. Thus, for example, intervening untranslated yet transcribed sequences can be present between a promoter sequence and the coding sequence and the promoter sequence can still be considered “operably linked” to the coding sequence.

Numerous examples of constitutive, cell-specific, and inducible promoters are known in the art, and one of skill could readily select a promoter for a specific intended use, e.g., the selection of the liver-specific human alpha-1 antitrypsin promoter for liver cell-specific expression, the selection of the constitutive CMV promoter for strong levels of continuous or near-continuous expression, or the selection of the inducible ecdysone promoter for induced expression. Induced expression allows the skilled artisan to control the amount of protein that is synthesized. In this manner, it is possible to vary the concentration of therapeutic product. Other examples of well known inducible promoters include: steroid promoters (e.g., estrogen and androgen promoters) and metallothionein promoters.

Gene expression can be enhanced by way of an “enhancer element.” By “enhancer element” is meant a DNA sequence (i.e., a cis-acting element) that, when bound by a transcription factor, increases expression of a gene relative to expression from a promoter alone. There are many enhancer elements known in the art, and the skilled artisan can readily select an enhancer element for a specific purpose. An example of an enhancer element useful for increasing gene expression in the liver is the apolipoprotein E hepatic control region (described in Schachter et al. (1993) J Lipid Res 34:1699-1707).

rAAV virions comprising a heterologous nucleic acid molecule may be introduced into the target organ for a variety of research and therapeutic uses. For example, DNA may be introduced to correct a defective gene that is expressed in the target organ. Additionally, DNA may be introduced to specifically delete or mutate a given gene by, for example, homologous recombination or to ascertain its function (functional genomics). Moreover, DNA may be introduced to express a protein. Such a protein may be expressed to achieve a therapeutic effect within the organism treated With rAAV. By “therapeutic effect” is meant an amelioration of a clinical sign or symptom of a disease or disorder. Alternatively, a protein may be expressed with the goal of isolating and purifying it, or of functionally characterizing it in vivo (functional proteomics).

The methods of the present invention generally provide for introducing rAAV virions into the liver of a mammal by way of (1) introducing a catheter into the liver and (2) infusing the rAAV virions through the catheter into the liver.

In a preferred embodiment, the rAAV virions are delivered by (1) creating an access site in an artery of a mammal, (2) introducing a guidewire into the artery, (3) introducing a sheath over the guidewire into the artery, (4) advancing a catheter through the sheath into the liver, (5) infusing the rAAV virions through the catheter into the liver, (6) removing the catheter and the sheath from the artery, and (7) repairing the access site.

In certain other embodiments, occluding one or more of the blood vessels delivering blood to, or emptying blood from, the liver is conducted. The occluding device can be, for example, a balloon attached to the tip of a catheter. In a preferred embodiment, all of the arteries and veins entering or leaving the liver are occluded so that rAAV virions are delivered by way of asanguinous hepatic perfusion.

In another embodiment, rAAV virions are delivered into the liver of the human by injection into the hepatic artery, wherein the rAAV virions contain a heterologous gene coding for a blood coagulation factor that is otherwise deficient or lacking in the human, such that cells within the liver express the heterologous gene at a level that provides a therapeutic effect. Such clinical signs or symptoms of a therapeutic effect include a reduction in whole blood clotting time, a reduction in activated partial thromboplastin time, or a reduction in exogenous coagulation factor usage. By “exogenous coagulation factor usage” is meant the administration (or self-administration) of purified or recombinant coagulation factor to treat or prevent the signs and/or symptoms of hemophilia.

The catheter may be introduced into the target organ through an appropriate vein or artery. For example, the liver may be accessed via either the portal vein or the hepatic artery. An appropriate vein or artery may be accessed using techniques known in the art, such as the Seldinger technique. See, e.g., Conahan et al., (1977) JAMA 237:446-447, herein incorporated by reference. Other methods of accessing veins and arteries are also known. See, e.g., U.S. Pat. No. 5,944,695, herein incorporated by reference.

The methods of the present invention may be used to deliver heterologous genes for the treatment of disorders that arise from or are related to liver cells and/or liver function. Such DNA and associated disease states include, but are not limited to: DNA encoding glucose-6-phosphatase, associated with glycogen storage deficiency type 1A; DNA encoding phosphoenolpyruvate-carboxykinase, associated with Pepck deficiency; DNA encoding galactose-1 phosphate uridyl transferase, associated with galactosemia; DNA encoding phenylalanine hydroxylase, associated with phenylketonuria; DNA encoding branched chain alpha-ketoacid dehydrogenase, associated with Maple syrup urine disease; DNA encoding fumarylacetoacetate hydrolase, associated with tyrosinemia type 1; DNA encoding methylmalonyl-CoA mutase, associated with methylmalonic acidemia; DNA encoding medium chain acyl CoA dehydrogenase, associated with medium chain acetyl CoA deficiency; DNA encoding ornithine transcarbamylase, associated with ornithine transcarbamylase deficiency; DNA encoding argininosuccinic acid synthetase, associated with citrullinemia; DNA encoding low density lipoprotein receptor protein, associated with familial hypercholesterolemia; DNA encoding UDP-glucouronosyltransferase, associated with Crigler-Najjar disease; DNA encoding adenosine deaminase, associated with severe combined immunodeficiency disease; DNA encoding hypoxanthine guanine phosphoribosyl transferase, associated with Gout and Lesch-Nyan syndrome; DNA encoding biotinidase, associated with biotinidase deficiency; DNA encoding beta-glucocerebrosidase, associated with Gaucher disease; DNA encoding beta-glucuronidase, associated with Sly syndrome; DNA encoding peroxisome membrane protein 70 kDa, associated with Zellweger syndrome; DNA encoding porphobilinogen deaminase, associated with acute intermittent porphyria; DNA encoding alpha-I antitrypsin for treatment of alpha-1 antitrypsin deficiency (emphysema); and DNA encoding a tumor suppessor gene such as p53 for the treatment of various cancers.

In certain embodiments, rAAV virions are used to deliver heterologous genes encoding “secretory proteins.” By “secretory proteins” is meant proteins or polypeptides that are secreted outside of the cell in which they were synthesized. Secretory proteins can be taken up by any cell (i.e., can become internally localized), including the cell in which they were synthesized, as long as they are first secreted outside of the cell in which they were synthesized. Alternatively, secretory proteins can be located to an extracellular compartment such as the extracellular matrix, the interstitial fluid, the surface of the skin, the lumen of an organ or blood vessel, or any other location not within or physically connected to a cell. By “blood vessel” is meant any vessel in the body that transports blood including, but not limited to, an artery, a vein, a venule, and a capillary.

Secretory proteins are not limited to those that are known to be naturally occurring, but encompass proteins not normally secreted in nature, which obtain the ability to be secreted by the incorporation of a signal sequence. Using well-known molecular biological techniques, the skilled artisan can insert a signal sequence in an appropriate location (usually 5′ to the start codon of a gene) within a plasmid or vector incorporating a gene, which, upon translation, enables a protein encoded therein to be secreted from the cell in which it was synthesized. Several signal sequences are known for a variety of proteins, all of which contain one or two positively charged amino acids followed generally by 6-12 hydrophobic residues (see, e.g., Leader, D. P. (1979) Trends Biochem. Sci. 4:205; Rapoport, T. A. (1985) Curr. Top. Membr. Transport 24:1-63).

The invention encompasses DNA encoding secretory proteins that include, but are not limited to, erythropoietin for treatment of anemia due to thalassemia or to renal failure; DNA encoding vascular endothelial growth factor, DNA encoding angiopoietin-1, and DNA encoding fibroblast growth factor for the treatment of ischemic diseases; DNA encoding tissue factor pathway inhibitor for the treatment of occluded blood vessels as seen in, for example, atherosclerosis, thrombosis, or embolisms; and DNA encoding a cytokine such as one of the various interleukins for the treatment of inflammatory and immune disorders, and cancers.

More preferably, the invention encompasses rAAV virions comprising heterologous genes encoding blood coagulation factor proteins, which proteins may be delivered, using the methods of the present invention, to the cells of a mammal having hemophilia for the treatment of hemophilia. Thus, the invention includes: delivery of delivery of the Factor VII gene for treatment of Factor VII , Factor VIII , Factor IX, or Factor XI deficiencies or Glanzmann thrombasthenia, delivery of the Factor X gene for treatment of Factor X deficiency, delivery of the Factor XI gene for treatment of Factor XI deficiency, delivery of the Factor XIII gene for treatment of Factor XIII deficiency, and, delivery of the Protein C gene for treatment of Protein C deficiency. Delivery of each of the above-recited genes to the cells of a mammal is accomplished by first generating a rAAV virion comprising the gene and then administering the rAAV virion to the mammal. Thus, the invention includes rAAV virions comprising genes encoding any one of Factor X, Factor VII, Factor XI, Factor XIII or Protein C.

Most preferably, the methods of the present invention encompass the delivery of Factor VIII for the treatment of hemophilia A and Factor IX (complete sequence available from GenBank Accession No. 182612) for the treatment of hemophilia B. Methods for generating human Factor VIII constructs suitable for incorporation in recombinant AAV vectors are described in U.S. Pat. Nos. 6,200,560, and 6,221,349, both herein incorporated by reference.

The following examples are given to illustrate various embodiments that have been made within the scope of the present invention. It is to be understood that the following examples are neither comprehensive nor exhaustive of the many types of embodiments that can be prepared in accordance with the present invention.

### EXAMPLE 1

### Hepatic Artery Infusion in Dogs

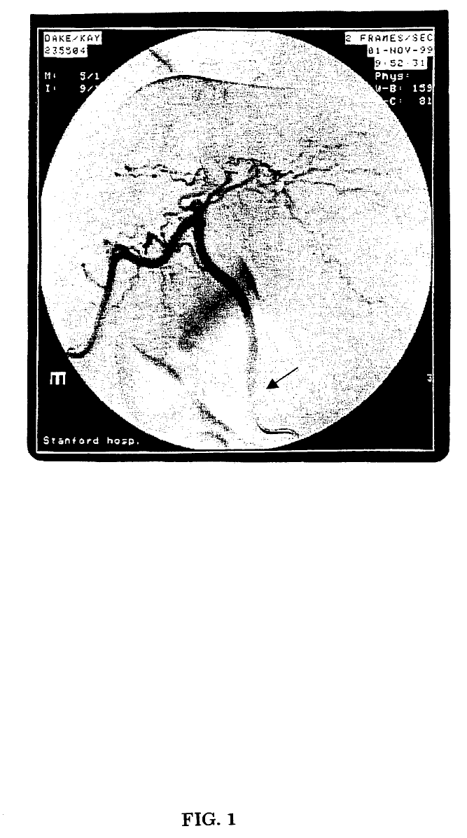

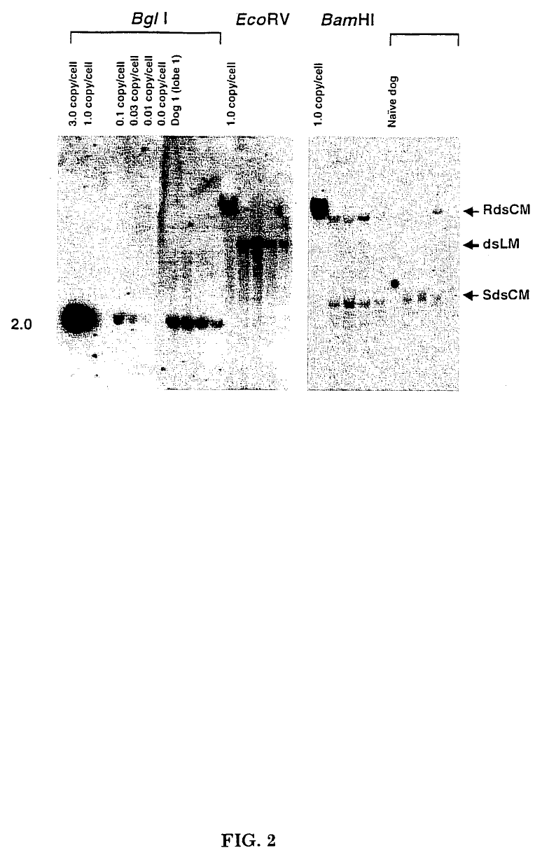

Three sexually mature male dogs were infused with rAAV-null vector via the hepatic artery at three separate doses: 3.7×10¹², 5.0×10¹², and 7.0×10¹² vg/kg. The hepatic artery perfusions were performed under fluoroscopy with general anesthesia. After sedation, the hepatic artery catheter was inserted in the femoral artery through to the aorta and then fed into the hepatic artery (see FIG. 1). The catheter has a single infusion point and a balloon proximal to the injection site. The balloon was inflated and the vector infused in ˜10 mL of the excipient over a three minute period. The catheter was washed with normal saline and left in place for another seven minutes at which time the balloon was deflated and the catheter removed. To identify gene transfer to the liver, DNA was extracted from all three dogs and subjected to Southern blot analysis. One dog had DNA extracted from four different liver lobes. Twenty μg of total DNA was digested with Bgl I and separated on 0.8% agarose gel. The separated DNA was blotted on a nylon membrane and hybridized to a ³²P-labeled 2.1-kb vector specific probe. Copy number standards (the number of double-stranded vector genomes per diploid genomic equivalent) were prepared by adding equivalent number of the vector plasmid molecules into 20 μg of total DNA extracted from naïve dog liver. All three dogs had detectable amounts of double-stranded rAAV genomes in their livers, establishing that quantifiable transduction occurred (see Table 1 and FIG. 2). The vector copy numbers range from 0.05 to 0.27 rAAV genomes per diploid amount of genomic DNA.

TABLE 1
Recombinant AAV Virion Transduction of Liver Lobes of Dogs

| Dog Number | Liver Lobe 1 | Liver Lobe 2 | Liver Lobe 3 | Liver Lobe 4 |
|---|---|---|---|---|
| HIK004 | 0.21* | 0.27 | ND** | ND |
| HF5612 | 0.13 | 0.05 | ND | ND |
| 235504 | 0.09 | 0.08 | 0.07 | 0.12 |

*Values are given in double-stranded copies per diploid cellular genomes.
**ND = Not Done.

### EXAMPLE 2

### Selective Lobe Infusion of Rat Livers

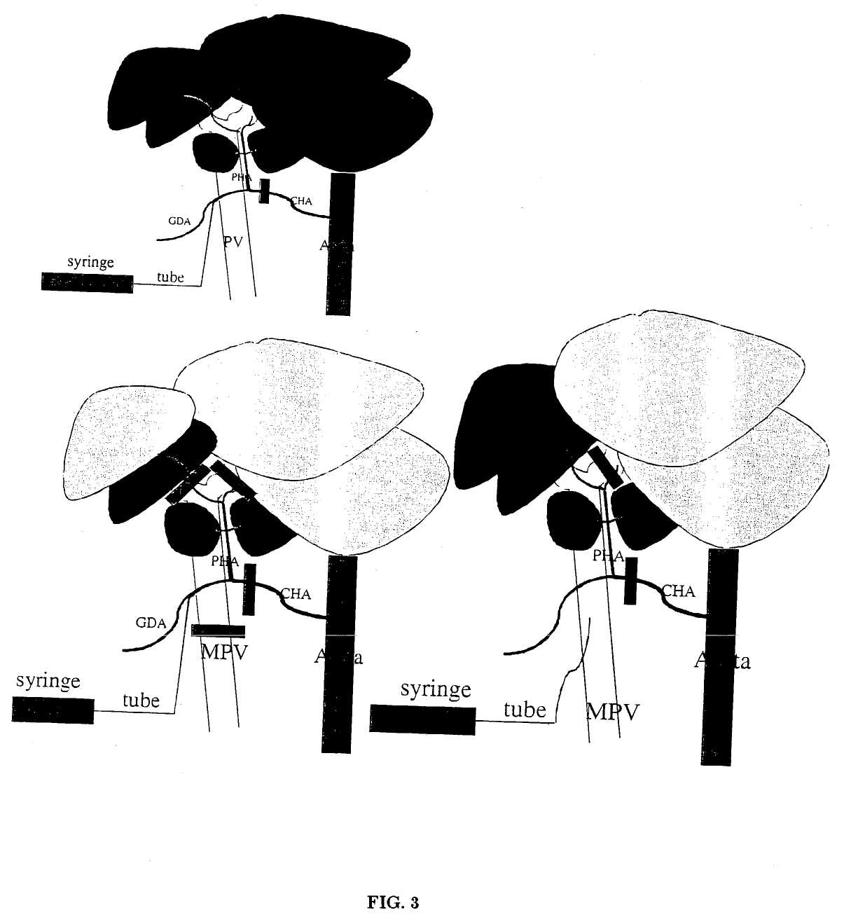

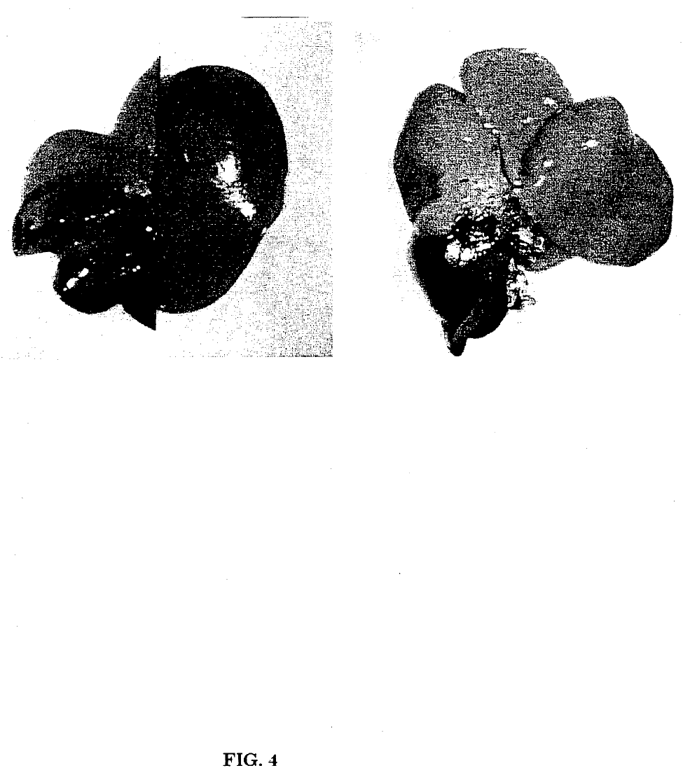

Rats (Lewis, weight 240 g) were infused with an injection into the left liver lobes using a portal vein approach (see FIG. 3). A laparotomy was performed with the aid of an operating microscope; the common hepatic artery (CHA), main portal vein (MPV), left portal vein (LPV), and right portal vein (RPV) were isolated from the surrounding tissues. After placing temporary clamps on the CHA and RPV together with the right hepatic artery (RHA), ink solution was injected by puncture of the MPV using a 30 gauge needle for 1 min. After finishing the injection, the needle was pulled out of the MPV. The other clamps were removed 5 minutes later. Only the left liver lobes (30% of the total liver mass) were stained black (see FIG. 4).

In a second set of infusions, the selected caudate lobes of the liver were infused by hepatic artery injection (see FIG. 3). A laparotomy was performed with the aid of an operating microscope, the CHA, hepatic artery branch for caudate lobes (HABC), proper hepatic artery (PHA) after branching the HABC, gastroduodenal artery (GDA), MPV, LPV, and RPV were isolated from the surrounding tissues. After placing a temporary clamp on the CHA, a PE10 tube was inserted in the GDA through the CHA at the branching of the HABC (see FIG. 3). To minimize leaking during injection, the tube was ligated over the artery. Temporary clamps were also placed on the MPV, LPV, and RPV. Ink solution was injected through the PE10 tube for 1 min. After the injection, the tube was removed, then the GDA was ligated at the proximal side of the tube insertion site. The clamp placed on the CHA was released, while the other clamps were removed 3 min later. Only the caudate liver lobes and inferior left lobe (15% of total liver mass) were stained black (see FIG. 4).

### EXAMPLE 3

### Asanguineous Hepatic Perfusion in Sheep (Ewes)

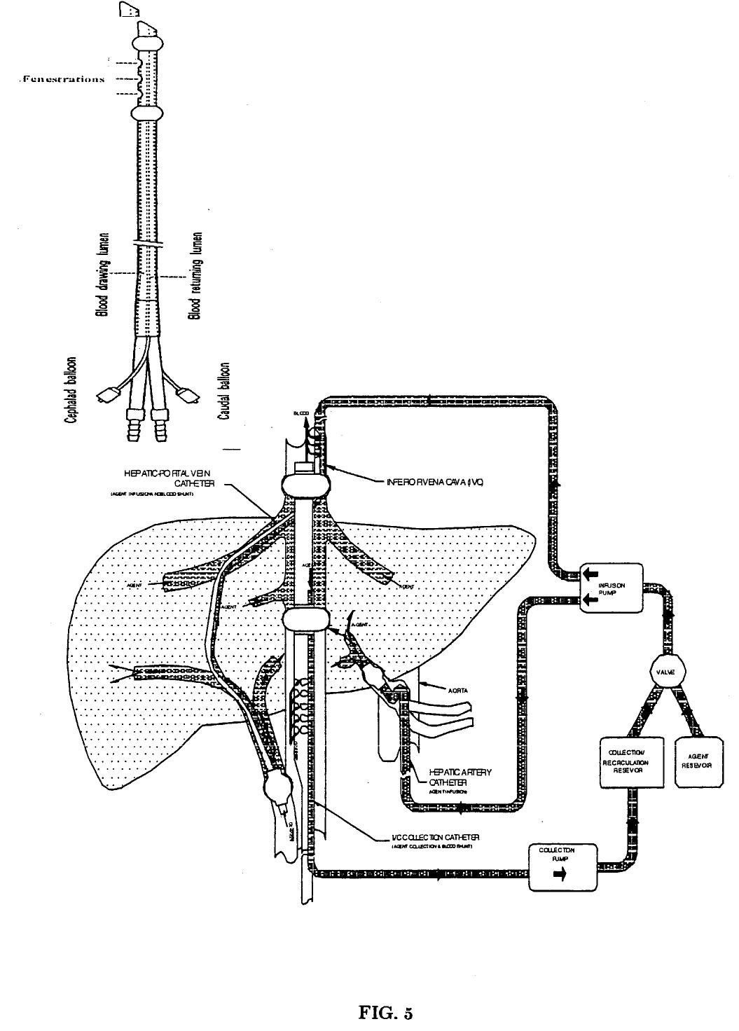

The right internal jugular vein is identified with portable ultrasonography. Seldinger technique catheterization of the vein is followed by insertion of a 0.035″ guidewire directed into the suprahepatic vena cava (superior to the right hepatic vein) under fluoroscopy and confirmed by hand-held angiography. A 12 Fr. Dilator is delivered into the jugular vein allowing the ultrasound cannula to be advanced through the sheath introducer and into the right hepatic vein. Following identification of a right portal vein target by duplex imaging, the trans-hepatic needle is loaded over a 0.018″ guide wire into the ultrasound cannula and advanced across the liver parenchyma and into the portal vein. If the portal vein has been accessed, a stiff 0.018″ guide wire is passed through the trans-hepatic needle and into the portal vein and the tract is dilated. The portal vein catheter is then delivered into the main portal vein. Blood shunting from the portal vein to the IVC/right atrium with inflow occlusion may occur. (See FIG. 5).

The right femoral vein is accessed by needle puncture below the inguinal ligament. Seldinger technique is employed to insert an 18 Fr. sheath to reach the IVC. Cannulation of the IVC to the right atrium is accomplished with 0.035″ super stiff wire or Cook “coat hanger.” Angiography of the IVC is performed to visualize all renal, adrenal, and hepatic veins. The IVC catheter is advanced cephalad whereby the tip is in the lower right atrium. The caudal balloon is positioned immediately cephalad to the right renal vein. Balloons may be inflated to assess hepatic venous isolation. Drainage of the hepatic venous effluent (blood or perfusate) is directed through an internal lumen to the reperfusion circuit as needed. To accomplish decompression of the lower vena cava circulation, this blood is extracted and redistributed (via the ex vivo collection system) to the right atrium (right jugular vein cannula). (See FIG. 5).

The left femoral artery is identified by hand-held ultrasonography and cannulated via Seldinger technique with a 7 Fr. sheath. Abdominal aortic angiographic is performed to determine hepatic arterialization. In the normal setting, the celiac trunk is cannulated with a 0.035″ flexible wire to the proximal proper hepatic artery. The hepatic artery catheter is then delivered into the distal proper hepatic artery. Angiography confirms the position of the catheter and its corresponding occlusion balloon. (See FIG. 5).

Bolus infusion of heparin sulfate (70U/kg) is used to prevent coagulation at the locus of all occlusion balloons. Halothane anesthesia is minimized due to impaired hepatic metabolism. The portal vein catheter occlusion balloon is inflated with 3 cm of saline to obstruct portal vein blood flow, but permit shunting internally. The hepatic artery catheter balloon is then inflated with 1 cm of water. The infusion pumps connected to the inflow vessels are then directed to deliver 500 mL of isotonic heparinized saline to evacuate the liver of all blood. The caudal and cephalad IVC balloons are then inflated sequentially to a pressure of 15 cm H₂O . Perfusate and blood are delivered into the right atrium. Internal shunting of lower vena cava blood is permitted at a flow rate of 500 mL per minute. Catheter tubing is inspected for the presence of blood within the isolated liver space. Blood pressure monitoring and central venous pressure measurement will be performed continuously. Hypotension, if it occurs, will be managed by dopamine infusion at 3 μg/kg/min, and volume loading with isotonic saline. Mean arterial pressure will be maintained at 60 mm Hg.

Perfusion of the isolated liver is then initiated with isotonic crystalloid tainted with methylene blue to determine whether systemic exposure is occurring as this will manifest within 2-5 min in collected urine. To determine whether biliary excretion occurs, a small upper midline laparotomy is performed following completion of AHP to aspirate bile from the gallbladder. Methylene blue discoloration of bile is also evident if loss of complete hepatic isolation occurs. Dwell time will not exceed 20 min, as previous studies have indicated that dwell time exceeding 20 min leads to hepatotoxicity. After incubation of the vector is complete, the vector perfusate is removed before blood flow to the liver is restored. Vector genome particle concentration is measured in the excipient prior to and after infusion in order to estimate the concentration of virus that remains in the liver.

Blood samples are obtained prior to vector delivery and then on days 1, 7, 14, and 30 for serum electrolytes, complete blood counts, renal function, and liver function tests including alkaline phosphatase, ALT, AST, GGT, and bilirubin. Neutralizing anti-AAV antibodies will also be monitored.

### EXAMPLE 4

### Hepatic Artery Infusion in Humans

To determine the feasibility of hepatic artery infusion of AAV-F.IX in humans having severe hemophilia B, a Phase I/II dose escalation study was initiated. As shown in Table 2, six subjects (subjects A-F) were treated at doses ranging from 8×10E10 to 2×10E12 vg/kg (vector titer determined by Q-PCR against linearized standard). All subjects were adult males with severe hemophilia B, with baseline Factor IX levels <1%, and all were HCV antibody positive. Those who were HCV RNA viral load positive underwent liver biopsy prior to enrollment and were enrolled only if the fibrosis score on the Metavir scoring system was F0-F2. Subjects were also enrolled only if they had greater than twenty exposure days of treatment with Factor IX protein and no history or presence of an inhibitor to Factor IX protein.

TABLE 2
Subject Demographics

| Patient | A | B | C | D | E | F |
|---|---|---|---|---|---|---|
| Age | 63 | 48 | 21 | 20 | 31 | 28 |
| (years) | | | | | | |
| Baseline | | | | | | |
| Factor IX | <1% | <1% | <1% | <1% | <1% | <1% |
| Mutation | R16 Stop | W310 Stop | N/A | R180Q | G133E | R4Q |
| CRM | | | | | | |
| Status* | Neg | Neg | N/A | Pos | Neg | Pos |
| Anti-AAV | 1:1-1:10 | <1:1 | 1:1-1:10 | 1:1-1:10 | 1:1-1:10 | 1:1-1:50 |
| Titer** | | | | | | |
| Dose | | | | | | |
| (vg/kg)*** | 8 × 10E10 | 8 × 10E10 | 4 × 10E11 | 4 × 10E11 | 2 × 10E12 | 2 × 10E12 |

*CRM (Cross-reacting material) Status refers to whether the individual expresses an endogenous polypeptide product that cross-reacts with Factor IX antibodies.
**Prior to treatment with AAV-F.IX.
***Titers based on Q-PCR (linearized standards).

AAV-hFIX16 Vector

rAAV containing the human Factor IX gene (“AAV-hFIX16”) was derived from the wild-type virus using standard recombinant DNA techniques. All of the viral genes were removed and replaced with the following: 1) an expression cassette that contains the human α1-antitrypsin promoter coupled to the human apolipoprotein E enhancer (for details see High K A, 1995, Adv Exper Med & Bio 386:79-86, incorporated by reference herein) and hepatocyte control region (for details see Nakai et al., 1999, J Virol 73: 5438-47, incorporated by reference herein); 2) exon 1 from the human Factor IX gene; 3) a portion of the human Factor IX intron 1 (for details see U.S. Pat. No. 6,093,392 incorporated by reference herein); 4) exons 2-8 of the human Factor IX gene; and 5) the bovine growth hormone polyadenylation signal sequence. Small intervening non-functional DNA sequences are derived in the process of assembling the genetic elements through recombinant DNA techniques. The expression cassette is flanked by the 145 nucleotide inverted terminal repeats derived from AAV type 2.

AAV-hFIX16 was supplied as a frozen liquid at a volume of 1 mL in 1.5 mL polypropylene sterile cryogenic screw cap vials and was stored at −20° C. or colder. Just prior to use, the frozen product was thawed at room temperature and gently mixed by flicking the vial ten times. The vial was tapped on the bench top two times to expel liquid present in the cap into the vial. The syringe for AAV-hFIX16 administration was prepared sterilely and filled in a biosafety hood. After filling, the syringe was stored in sterile bags on wet ice. The syringe was warmed to room temperature just prior to infusion. The product was administered within two hours of thawing to assure maximum potency.

Angiographic Procedure

Using the standard Seldinger technique, the common femoral artery was cannulated with an angiographic introducer sheath. The patient was then heparinized by IV injection of 100U/kg of heparin. A pigtail catheter was then advanced into the aorta and an abdominal aortogram was performed. Following delineation of the celiac and hepatic arterial anatomy, the proper HA was selected using a standard selective angiography catheter (Simmons, Sos-Omni, Cobra or similar catheters). Prior to insertion into the patient, all catheters were flushed with normal saline. Selective arteriogram was then performed using a non-ionic contrast material (Omnipaque or Visipaque). The catheter was removed over a 0.035 wire (Bentsen, angled Glide, or similar wire). A 6F Guide-sheath (or guide catheter) was then advanced over the wire into the common HA. The wire was then exchanged for a 0.018 wire (FlexT, Microvena Nitenol, or similar wire) and a 6×2 Savvy balloon was advanced over the wire into the proper HA distal to the gastrodoudenal artery. The wire was then removed, the catheter tip position confirmed by hand injection of contrast into the balloon catheter, and the lumen flushed with 15 ml of heparinized normal saline (NS) to fully clear the contrast. Prior to infusion of the AAV-hFIX16, the balloon was inflated to 2 atm to occlude the flow lumen of the HA. AAV-hFIX16, at the doses shown in Table 2, was brought to a final volume of approximately less than or equal to 40 ml (depending on dose and weight of patient) and was then infused over 10-12 minutes using an automatic volumetric infusion pump. Three milliliters (ml) of normal saline (NS) was then infused (at the same rate as the AAV-hFIX16), to clear the void volume of the catheter. The balloon remained inflated for 2 minutes at which time the balloon was deflated and the catheter removed. A diagnostic arteriogram of the femoral puncture site was then performed in the ipsilateral anterior oblique projection. The puncture site was closed by standard methods, e.g., utilizing a percutaneous closure device using either a 6 F Closer (Perclose Inc., Menlo Park, Calif.) or a 6 F Angioseal, or by manual compression applied for 15 to 30 minutes at the site of catheter removal.

Results

The 4 subjects treated in the first two dose cohorts showed no vector-related toxicity but failed to achieve Factor IX levels above baseline. Subject E, the first to receive a dose of 2×10E12 vg/kg, showed circulating F.IX levels in the range of 5-12%, first detected two weeks after vector infusion (and one week after the last F.IX protein infusion) and present continuously over the ensuing three weeks. Six weeks after infusion, the levels fell to 2.7%. Subject F, also treated at 2×10E12 vg/kg, showed no toxicity after vector infusion, but the highest F.IX level measured was 3%, determined 1 week after the last dose of exogenous Factor IX. At two weeks post-injection, anti-AAV antibody titers were at 1:10³-1:10⁴ for Subjects A, C, and E; 1:10-1:10³ for Subjects B and D; and 1:10³ for Subject F. This study demonstrates that AAV-F.IX delivered via hepatic artery infusion to the liver can transduce human hepatocytes in vivo and lead to the expression of therapeutic levels of Factor IX.

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

We claim:

1. A method for delivering a heterologous nucleic acid molecule of interest to the liver of a human, comprising the steps of:
(a) providing a preparation of rAAV virions wherein said rAAV virions comprise a heterologous nucleic acid molecule of interest;
(b) introducing a catheter into a blood vessel of said human;
(c) threading said catheter through the vasculature to the liver of said human; and
(d) infusing said rAAV virions through said catheter into the liver such that said liver is transduced by said rAAV virions and said heterologous nucleic acid molecule is expressed.

2. The method of claim 1, wherein the catheter is introduced into the liver via an artery.

3. The method of claim 2, wherein said catheter is introduced into the liver via a hepatic artery.

4. The method of claim 3, wherein said artery is accessed through an opening in a femoral artery.

5. The method of claim 1, wherein the heterologous nucleic acid molecule encodes a blood coagulation factor.

6. The method of claim 5, wherein the coagulation factor is Factor IX.

7. The method of claim 6, wherein said Factor IX is human Factor IX.

8. The method of claim 7, wherein the blood coagulation factor is expressed at levels providing for a therapeutic effect.

9. The method of claim 8, wherein said therapeutic effect is a reduction in whole blood clotting time.

10. The method of claim 8, wherein said therapeutic effect is a reduction in activated prothromboplastin time.

11. The method of claim 8, wherein said therapeutic effect is a reduction in exogenous coagulation factor usage.
